(19) United States
(12) Patent Application Publication
(10) Pub. No.: US 2023/0091629 A1
(43) Pub. Date: Mar. 23, 2023

(54) STENT SYSTEM

(71) Applicant: BOSTON SCIENTIFIC SCIMED, INC., Maple Grove, MN (US)

(72) Inventors: Ryan V. Wales, Northborough, MA (US); Paul Smith, Smithfield, RI (US); Morgan Zhu, Somerville, MA (US); Jay Gray, Sudbury, MA (US); Laura Elizabeth Christakis, Framingham, MA (US)

(73) Assignee: BOSTON SCIENTIFIC SCIMED, INC., Maple Grove, MN (US)

(21) Appl. No.: 17/941,754

(22) Filed: Sep. 9, 2022

Related U.S. Application Data
(60) Provisional application No. 63/245,241, filed on Sep. 17, 2021.

(51) Int. Cl. A61F 2/95; A61F 2/82

(52) U.S. Cl. CPC A61F 2/95; A61F 2/82

(57) ABSTRACT

A stent system may include a stent including a first leg having a first end fixedly attached to the distal end of a main body portion and extending distally from the distal end of the main body portion in a deployed configuration, and a second leg having a first end fixedly attached to the distal end of the main body portion and extending distally from the distal end of the main body portion in the deployed configuration. The second leg may extend proximally from the distal end of the main body portion in a delivery configuration. A stent system may include a bifurcated delivery sheath and two guidewires for delivery of two stents at the same time. A method of treating a body lumen may include delivering a contrast fluid including an anti-gas agent while implanting a stent in the body lumen.

### CROSS-REFERENCE TO RELATED APPLICATIONS

This application claims the benefit of U.S. Provisional patent application Ser. No. 63/245,241, filed Sep. 17, 2021, which is incorporated herein by reference.

### TECHNICAL FIELD

The present disclosure pertains to medical devices, and methods for manufacturing and/or using medical devices. More particularly, the present disclosure pertains to an improved design for an endoprosthesis or stent.

### BACKGROUND

Current braided or knitted self-expanding stents may express a large degree of longitudinal flexibility due to design and device length. This may be advantageous for the purpose of device delivery, especially in more tortuous anatomical regions and for reduction in lumen straightening post-delivery, which is typically seen as being less traumatic on target lumens. Uncovered metallic endoprostheses or stents are sometimes placed for chronic conditions but are generally not removable. Plastic endoprostheses or stents may be prone to blockage which may require repeat treatment(s) and are sometimes unable to open the stricture that initially caused the blockage of the affected body lumen (e.g., the bile duct, the pancreatic duct, etc.). Additionally, the biliary tree has several branches, bifurcations, and/or adjoining lumens. Placement of an endoprosthesis or stent within or across a bifurcation may present additional and/or different challenges. Conventionally, placing endoprostheses or stents at a bifurcation may require several steps, devices, introductions, removals, and/or exchanges of instruments, connections and/or disconnections by fluid management devices, etc. In some cases, air bubbles may be introduced into the surgical instruments, tools, and/or the body lumen being treated. Air bubbles are undesirable as they may obstruct the physician's visibility and/or they may present risk to the patient. There is an ongoing need to provide alternative endoprosthesis or stent systems as well as alternative methods for manufacturing and using endoprosthesis or stent systems.

### SUMMARY

In one example, a stent system may comprise a stent configured to shift between a delivery configuration and a deployed configuration, the stent comprising a main body portion having a proximal end and a distal end, a first leg having a first end fixedly attached to the distal end of the main body portion and a second end opposite the first end, the first leg extending distally from the distal end of the main body portion in the deployed configuration, and a second leg having a first end fixedly attached to the distal end of the main body portion and a second end opposite the first end, the second leg extending distally from the distal end of the main body portion in the deployed configuration. The second leg extends proximally from the distal end of the main body portion in the delivery configuration.

In addition or alternatively to any example described herein, the second leg is inverted into the main body portion such that the second leg extends proximally from the distal end of the main body portion within the main body portion in the delivery configuration.

In addition or alternatively to any example described herein, the stent system may comprise an elongate shaft having a lumen extending therein. The stent may be disposable within the lumen in the delivery configuration. The stent may be configured to shift from the delivery configuration to the deployed configuration when the stent is disposed outside of the lumen.

In addition or alternatively to any example described herein, the stent system may comprise a first mandrel slidably disposed within the lumen, and a second mandrel slidably disposed within the lumen alongside the first mandrel. The first mandrel is at least partially disposed within the first leg and the second mandrel is at least partially disposed within the second leg.

In addition or alternatively to any example described herein, a first guidewire is slidably disposed within a first lumen extending within the first mandrel.

In addition or alternatively to any example described herein, a second guidewire is slidably disposed within a second lumen extending within the second mandrel.

In addition or alternatively to any example described herein, the second mandrel is configured to shift the stent from the delivery configuration toward the deployed configuration.

In addition or alternatively to any example described herein, the second mandrel includes a distally facing shoulder configured to engage the second end of the second leg in the delivery configuration such that distal advancement of the second mandrel everts the second leg to shift the stent toward the deployed configuration.

In addition or alternatively to any example described herein, a distal portion of the first mandrel has a D-shaped cross-section having a first flat side, a distal portion of the second mandrel has a D-shaped cross-section having a second flat side, and the first flat side faces the second flat side within the lumen of the elongate shaft.

In addition or alternatively to any example described herein, the first leg is tapered radially inward from the first end toward the second end.

In addition or alternatively to any example described herein, the second leg is tapered radially inward from the first end toward the second end.

In addition or alternatively to any example described herein, a stent system may comprise a delivery sheath including a main body portion having a proximal end and a distal end, a first leg having a first end fixedly attached to the distal end of the main body portion and a second end opposite the first end, and a second leg having a first end fixedly attached to the distal end of the main body portion and a second end opposite the first end, a first mandrel slidably disposed within the first leg of the delivery sheath, a second mandrel slidably disposed within the second leg of the delivery sheath, a first guidewire slidably disposed within the first mandrel, and a second guidewire slidably disposed within the second mandrel.

In addition or alternatively to any example described herein, the delivery sheath includes a first lumen extending from the proximal end of the main body portion to the second end of the first leg and a second lumen extending from the proximal end of the main body portion to the second end of the second leg.

In addition or alternatively to any example described herein, the first lumen has a D-shaped cross-section having a first flat side and the second lumen has a D-shaped cross-section having a second flat side.

In addition or alternatively to any example described herein, the first lumen and the second lumen share a common wall defining both the first flat side and the second flat side.

In addition or alternatively to any example described herein, the first leg has a flat side, and the second leg has a flat side facing toward the flat side of the first leg.

In addition or alternatively to any example described herein, the stent system may comprise an elongate shaft having a lumen extending therein. The delivery sheath may be disposable within the lumen of the elongate shaft and axially slidable with respect to the elongate shaft. The flat side of the first leg matingly engages the flat side of the second leg when the first leg and the second leg are disposed within the lumen of the elongate shaft.

In addition or alternatively to any example described herein, the stent system may comprise a first stent disposable within the first leg distal of the first mandrel, and a second stent disposable within the second leg distal of the second mandrel. The first mandrel is configured to push the first stent out of the first leg via axial translation of the first mandrel relative to the first leg. The second mandrel is configured to push the second stent out of the second leg via axial translation of the second mandrel relative to the second leg.

In addition or alternatively to any example described herein, the second end of the first leg and the second end of the second leg are biased away from each other laterally with respect to a longitudinal axis of the main body portion.

In addition or alternatively to any example described herein, a stent system may comprise an elongate shaft configured to access a body lumen of a patient, the elongate shaft having a lumen extending therein, a delivery device slidably disposed within the lumen, the delivery device being configured to deliver a stent to the body lumen, and a source of contrast fluid in fluid communication with the elongate shaft for delivery to the body lumen. The contrast fluid may include an anti-gas agent.

In addition or alternatively to any example described herein, a method of treating a body lumen may comprise accessing a body lumen of a patient with an elongate shaft having a lumen extending therein, inserting a delivery device within the lumen of the elongate shaft, the delivery device being configured to deliver a stent to the body lumen, and delivering a contrast fluid including an anti-gas agent while implanting the stent within the body lumen.

The above summary of some embodiments, aspects, and/or examples is not intended to describe each disclosed embodiment or every implementation of the present disclosure. The figures and detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 illustrate aspects of a stent;

FIGS. 3-9 illustrate aspects of a stent system and a method of using the stent system;

FIG. 10 illustrates aspects of a stent system;

FIG. 10A is cross-sectional view of a portion of the stent system of FIG. 10;

FIGS. 11-15 illustrate aspects of a stent system of FIG. 10 and a method of using the stent system; and

FIG. 16 is block diagram depicting aspects of a stent system and a method of using the stent system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of an endoprosthesis or stent system. It should be noted that in any given figure, some features of the endoprosthesis or stent system may not be shown, or may be shown schematically, for simplicity. Additional details regarding some of the components of the endoprosthesis or stent system may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the filament”, “the cell”, or other features may be equally referred to all instances and quantities beyond one of said feature. As such, it will be understood that the following discussion may apply equally to any and/or all of the components for which there are more than one within the endoprosthesis or stent system, unless explicitly stated to the contrary. Additionally, not all instances of some elements or features may be shown in each figure for clarity.

The following disclosure describes aspects of a stent system. In the interest of clarity and/or brevity, the term “stent” shall be used herein, and the term “stent” shall include and/or encompass other similar terms of art such as but not limited to “endoprosthesis”, etc. The disclosure also refers to treatment of a body lumen, and in particular a body lumen having a bifurcation and/or adjacent branches. In the interest of brevity, the term “body lumen” shall include but not be limited to such specific body lumens as the biliary tree, the hepatic ducts, the cystic duct, the common bile duct, the pancreatic duct, the bronchi, etc. The system is also contemplated for use in other body lumens.

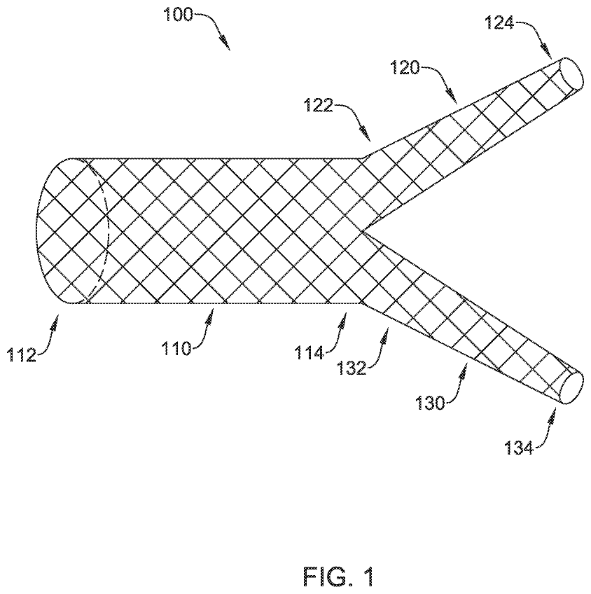

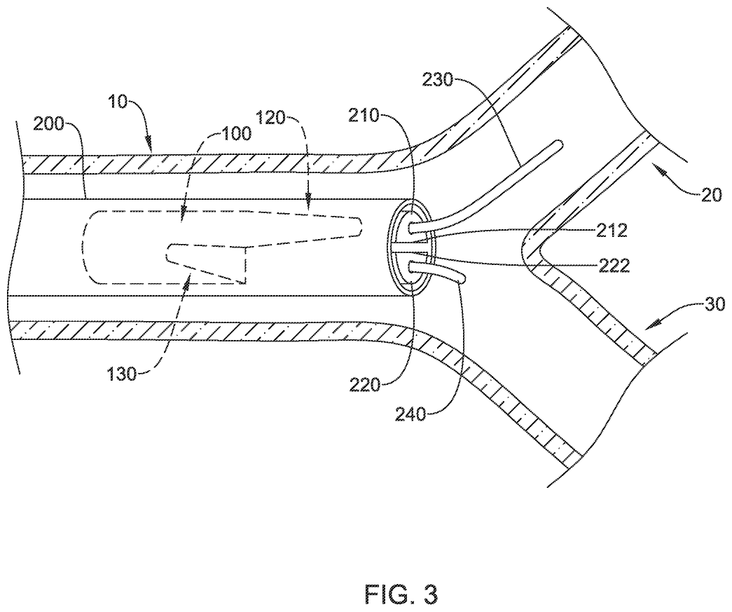

FIG. 1 illustrates a stent 100 comprising an expandable framework. The stent 100 and/or the expandable framework may be configured to shift between a radially contracted, delivery configuration and a radially expanded, deployed configuration. The delivery configuration may be a configuration in which the stent 100 is axially elongated and/or radially collapsed or compressed compared to the deployed configuration. The deployed configuration may be a configuration in which the stent 100 is axially shortened and/or radially expanded compared to the delivery configuration. In at least some embodiments, the stent 100 and/or the expandable framework may be self-expandable. For example, the stent 100 and/or the expandable framework may be formed from a shape memory material, such as nitinol. In some embodiments, the stent 100 and/or the expandable framework may be mechanically expandable. For example, the stent 100 and/or the expandable framework may be expandable using an inflatable balloon, using an actuation member, or other suitable means. During delivery to a treatment site, the stent 100 and/or the expandable framework may be disposed within a lumen of an elongate shaft (e.g., FIG. 3) in the delivery configuration. Upon removal from the lumen of the elongate shaft, the stent 100 and/or the expandable framework may shift and/or may be shifted from the delivery configuration to the deployed configuration.

The expandable framework may include and/or be formed with a plurality of cells. In some embodiments, the expandable framework may include one or more filaments interwoven to form the stent 100 and/or the expandable framework. In at least some embodiments, the one or more filaments may form and/or define the plurality of cells. In some embodiments, the expandable framework may be braided, knitted, or woven from the one or more filaments. In some embodiments, the one or more filaments may be wires, threads, strands, etc. In some embodiments, adjacent filaments of the one or more filaments may define the cells (i.e., openings or interstices) through a wall of the expandable framework. Alternatively, in some embodiments, the expandable framework may be a monolithic structure formed from a cylindrical tubular member, such as a single, cylindrical laser-cut nickel-titanium (e.g., nitinol) tubular member, in which the remaining (e.g., unremoved) portions of the tubular member form the stent 100 and/or the expandable framework with cells (i.e., openings or interstices) defined therein.

In some embodiments, the stent 100 and/or the expandable framework may be sufficiently flexible to permit the stent and/or the expandable framework to invert and/or fold over on or within itself in the delivery configuration. As such, at least a portion of the stent 100 and/or the expandable framework may be invertible in the delivery configuration.

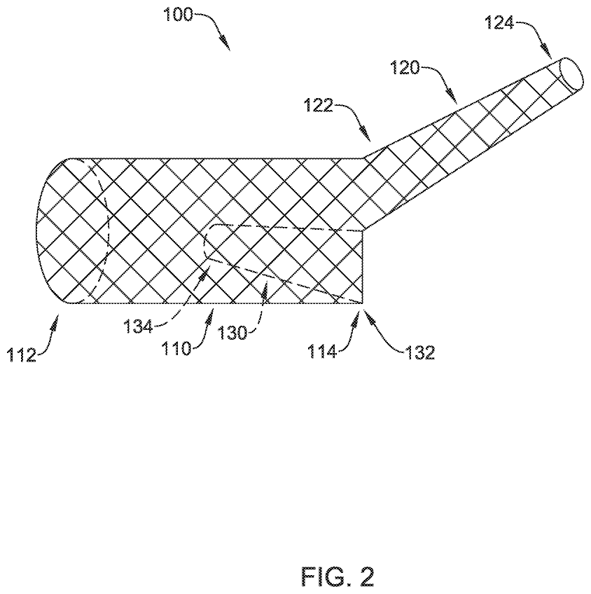

In some embodiments, the stent 100 and/or the expandable framework may include a main body portion 110 having a proximal end 112 and a distal end 114. In some embodiments, the stent 100 and/or the expandable framework may be a bifurcated stent including a first leg 120 and a second leg 130 extending from the main body portion 110. For example, the first leg 120 may have a first end 122 fixedly attached to the distal end 114 of the main body portion 110 and a second end 124 opposite the first end 122. The first leg 120 may extend distally from the distal end 114 of the main body portion 110 in the deployed configuration. In at least some embodiments, the first leg 120 may extend distally from the distal end 114 of the main body portion 110 in the delivery configuration. The second leg 130 may have a first end 132 fixedly attached to the distal end 114 of the main body portion 110 and a second end 134 opposite the first end 132. The second leg 130 may extend distally from the distal end 114 of the main body portion 110 in the deployed configuration. In at least some embodiments, the second leg 130 may extend proximally from the distal end 114 of the main body portion 110 in the delivery configuration. In some embodiments, the second leg 130 may be invertible and/or may be inverted into the main body portion 110 such that the second leg 130 extends proximally from the distal end 114 of the main body portion 110 within the main body portion 110 in the delivery configuration, as seen in FIG. 2.

In some alternative embodiments, the first leg 120 may extend proximally from the distal end 114 of the main body portion 110 in the delivery configuration. In some embodiments, the first leg 120 may be invertible and/or may be inverted into the main body portion 110 such that the first leg 120 extends proximally from the distal end 114 of the main body portion 110 within the main body portion 110 in the delivery configuration.

In some alternative embodiments, the first leg 120 and the second leg 130 may both extend proximally from the distal end 114 of the main body portion 110 in the delivery configuration. In some embodiments, the first leg 120 and the second leg 130 may both be invertible and/or may both be inverted into the main body portion 110 such that the first leg 120 and the second leg 130 both extend proximally from the distal end 114 of the main body portion 110 within the main body portion 110 in the delivery configuration.

In some embodiments, the expandable framework, the main body portion 110, the first leg 120, and/or the second leg 130 may be substantially tubular and/or may include and/or define at least one lumen extending axially therein. For instance the first leg 120 may include a lumen extending therethrough and the second leg 130 may include a lumen extending therethrough. The lumen of the first leg 120 may converge with the lumen of the second leg 130 at the distal end 114 of the main body portion 110 such that the lumens of the first leg 120 and the second leg 130 merge with the lumen of the main body portion 110 at the distal end 114 of the main body portion 110. In some embodiments, the expandable framework may have an axial length of about 25 millimeters to about 250 millimeters, about 40 millimeters to about 225 millimeters, about 60 millimeters to about 200 millimeters, about 80 millimeters to about 175 millimeters, about 100 millimeters to about 150 millimeters, or another suitable range. In some embodiments, the expandable framework may have a radial outer dimension or radial extent of about 3 millimeters to about 30 millimeters, about 5 millimeters to about 25 millimeters, about 6 millimeters to about 20 millimeters, about 8 millimeters to about 15 millimeters, or another suitable range. In some embodiments, the first leg 120 may have a first radial outer dimension, the second leg 130 may have a second radial outer dimension, and the main body portion 110 may have a third radial outer dimension greater than the first radial outer dimension and/or the second radial outer dimension. Other configurations are also contemplated. Some suitable but non-limiting materials for the stent 100, the expandable framework, and/or components or elements thereof, for example metallic materials and/or polymeric materials, are described below.

In some embodiments, the first radial outer dimension of the first leg 120 may be tapered radially inward from the first end 122 of the first leg 120 toward and/or to the second end 124 of the first leg 120 in the delivery configuration and/or the deployed configuration. In some embodiments, the second radial outer dimension of the second leg 130 may be tapered radially inward from the first end 132 of the second leg 130 toward and/or to the second end 134 of the second leg 130 in the delivery configuration and/or the deployed configuration. In some embodiments, the first leg 120 may be tapered radially inward from the first end 122 of the first leg 120 toward and/or to the second end 124 of the first leg 120 and the second leg 130 may be tapered radially inward from the first end 132 of the second leg 130 toward and/or to the second end 134 of the second leg 130 in the delivery configuration and/or the deployed configuration. In some embodiments, the third radial outer dimension of the main body portion 110 may be substantially constant from the proximal end 112 to the distal end 114 in the delivery configuration and/or the deployed configuration. In some embodiments, the main body portion 110 may be tapered radially inward from the proximal end 112 of the main body portion 110 toward and/or to the distal end 114 of the main body portion 110 in the delivery configuration and/or the deployed configuration. Other configurations are also contemplated.

In at least some embodiments, the stent 100 and/or the expandable framework may be disposed within the body lumen extending through a stricture to maintain and/or re-establish patency of the body lumen. In some embodiments, the stent 100 and/or the expandable framework may be configured to dilate at least a portion of the body lumen in the deployed configuration. For example, the stent 100 and/or the expandable framework may be configured to exert a radially outward force upon a wall of the body lumen and/or against a stricture that has formed therein.

In some embodiments, the stent 100 and/or the expandable framework may include a flared portion proximate the proximal end 112 of the main body portion 110. The flared portion may extend from the proximal end 112 toward the distal end 114. In some embodiments, the flared portion may have a generally constant outer diameter along its length. Other configurations are also contemplated, including but not limited to, a constant taper along the flared portion. In some embodiments, the outer diameter of the flared portion may be greater than the third radial outer dimension of the main body portion 110.

In some embodiments, the stent 100 may include a polymeric cover (not shown) disposed on and/or over at least a portion of the expandable framework (e.g., the main body portion 110, the first leg 120, the second leg 130, etc.). In some embodiments, the polymeric cover may be disposed on and/or along an outer surface of the expandable framework. In some embodiments, the expandable framework may be embedded in the polymeric cover. In some embodiments, the polymeric cover may be fixedly or releasably secured to, bonded to, or otherwise attached to the expandable framework. In some embodiments, the polymeric cover may be impermeable to fluids, debris, medical instruments, etc. In some embodiments, one or more portions of the expandable framework may be devoid of the polymeric cover. Some suitable but non-limiting materials for the polymeric cover are described below.

In some embodiments, to aid in positioning the stent 100 within a body lumen, the stent 100 may include at least one radiopaque marker disposed on and/or along the expandable framework. Some suitable but non-limiting materials for the at least one radiopaque marker are described below.

In use, when the stent 100 is positioned within a body lumen with the stent 100 and/or the expandable framework in the deployed configuration, the polymeric cover disposed on and/or over the expandable framework may form a barrier, such as a sealed interface, between the lumen of the stent 100 and/or the expandable framework and the wall of the body lumen positioned radially outward of the polymeric cover. The polymeric cover may isolate the lumen of the stent 100 and/or the expandable framework from the wall of the body lumen. The polymeric cover may prevent tissue ingrowth into the lumen and/or the expandable framework of the stent 100 and thereby permit and/or aid removal of the stent 100 and/or the expandable framework from the body lumen.

In some alternative embodiments and/or uses, implantation of the stent 100 may be permanent and/or may not be intended to be removed. In some such embodiments and/or uses, at least a portion of the expandable framework may be devoid of the polymeric cover so as to promote tissue ingrowth and thereby prevent migration of the stent 100 within the body lumen.

FIGS. 3-9 illustrate aspects of a stent system in use within a body lumen 10. As seen in FIG. 3, the body lumen 10 may include a first branch lumen 20 and a second branch lumen 30 fluidly connected to the body lumen 10 at a Y-junction. In some embodiments, the first branch lumen 20 and the second branch lumen 30 may form and/or define a bifurcation of the body lumen 10.

The stent system may include the stent 100, shown in phantom. In some embodiments, the stent system may include an elongate shaft 200 having a lumen extending therein. The stent 100 may be disposable and/or may be disposed within the lumen in the delivery configuration, as shown in FIG. 3. The stent 100 may be configured to shift from the delivery configuration to the deployed configuration when the stent 100 is disposed outside of the lumen of the elongate shaft 200 and/or when the stent 100 is no longer constrained by the elongate shaft 200.

The stent system may further include a first mandrel 210 slidably disposed within the lumen of the elongate shaft 200. The stent system may further include a second mandrel 220 slidably disposed within the lumen of the elongate shaft 200 alongside the first mandrel 210. In some embodiments, the first mandrel 210 and/or the second mandrel 220 may extend proximally to a proximal end of the elongate shaft 200 to be manipulated by the user. In some alternative embodiments, the first mandrel 210 and/or the second mandrel 220 may extend to an actuation mechanism disposed proximate the proximal end of the elongate shaft 200, wherein the actuation mechanism is configured to translate the first mandrel 210 and/or the second mandrel 220 axially relative to the elongate shaft 200.

The first mandrel 210 may be at least partially disposed within the main body portion 110 and extend into the first leg 120 of the stent 100 in the delivery configuration. The second mandrel 220 may be at least partially disposed within the main body portion 110 (e.g., alongside the first mandrel 210) and extend into the second leg 130 of the stent 100 in the delivery configuration. In at least some embodiments, the second mandrel 220 may be at least partially disposed within the second leg 130 of the stent 100 when the second leg 130 is inverted within the main body portion 110 of the stent 100. In some embodiments, the first mandrel 210 may be at least partially disposed within the first leg 120 of the stent 100 when the first leg 120 is inverted within the main body portion 110 of the stent 100. In some embodiments, the first mandrel 210 may extend distally of the first leg 120 of the stent 100. In some embodiments, the second mandrel 220 may extend distally of the second leg 130 of the stent 100.

As seen in FIG. 3, a distal portion of the first mandrel 210 may have a D-shaped cross-section having a first flat side 212 and a distal portion of the second mandrel 220 may have a D-shaped cross-section having a second flat side 222. The first flat side 212 may face the second flat side 222 within the lumen of the elongate shaft 200 and/or when the distal portion of the first mandrel 210 and the distal portion of the second mandrel 220 are both positioned within the lumen of the elongate shaft 200. In some embodiments, the first flat side 212 may extend along substantially an entire length of the first mandrel 210. In some embodiments, the first flat side 212 may extend proximally from a distal end of the first mandrel 210 to a medial portion of the first mandrel 210, where the first flat side 212 may taper outwardly from a central axis of the first mandrel 210 until the first flat side 212 ends and/or effectively disappears at an outer surface of the first mandrel 210 proximal of the stent 100. In some embodiments, the second flat side 222 may extend along substantially an entire length of the second mandrel 220. In some embodiments, the second flat side 222 may extend proximally from a distal end of the second mandrel 220 to a medial portion of the second mandrel 220, where the second flat side 222 may taper outwardly from a central axis of the second mandrel 220 until the second flat side 222 ends and/or effectively disappears at an outer surface of the second mandrel 220 proximal of the stent 100. The first flat side 212 on the distal portion of the first mandrel 210 and the second flat side 222 of the distal portion of the second mandrel 220 may permit the first mandrel 210 and the second mandrel 220 to take up less combined space within the lumen of the elongate shaft 200 proximal a distal end of the elongate shaft 200 such that the lumen of the elongate shaft 200 may accommodate the stent 100 therein within any increase in size and/or without a distally flared end on the elongate shaft 200.

In some embodiments, the first leg 120 of the stent 100 may conform to an outer shape and/or profile of the distal portion of the first mandrel 210. In some embodiments, the first leg 120 may be configured to stretch around the distal portion of the first mandrel 210 and/or may be configured to assume a similar shape (e.g., a D-shaped cross-section) as the first mandrel 210. In some embodiments, the second leg 130 of the stent 100 may conform to an outer shape and/or profile of the distal portion of the second mandrel 220. In some embodiments, the second leg 130 may be configured to stretch around the distal portion of the second mandrel 220 and/or may be configured to assume a similar shape (e.g., a D-shaped cross-section) as the second mandrel 220.

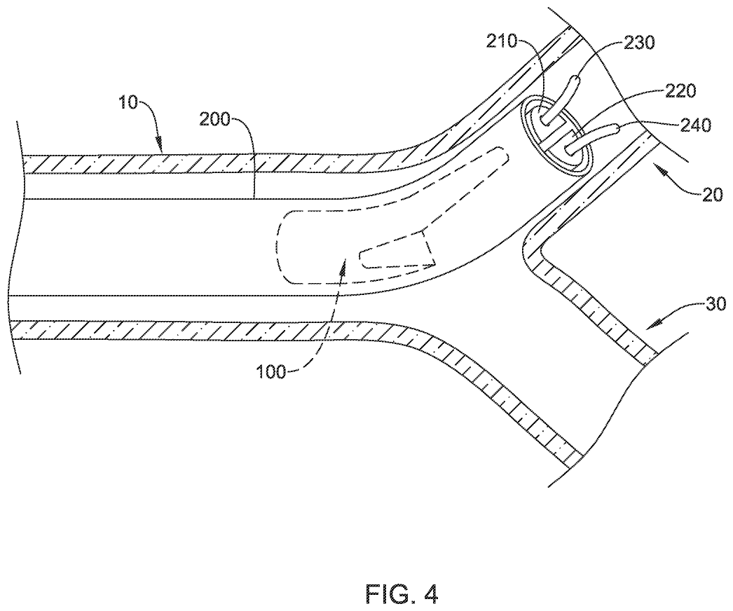

In some embodiments, the stent system may include a first guidewire 230 slidably disposed within a first lumen extending within the first mandrel 210. The first mandrel 210 may be configured to slide along and/or track over the first guidewire 230 within the body lumen 10. In some embodiments, the first guidewire 230 may be extended out of the first lumen of the first mandrel 210 and advanced into the first branch lumen 20. Thereafter, the first mandrel 210 and the elongate shaft 200 (and the stent 100 positioned therein) may be advanced and/or tracked over the first guidewire 230 into the first branch lumen 20, as shown in FIG. 4.

Returning briefly to FIG. 3, in some embodiments, the stent system may include a second guidewire 240 slidably disposed within a second lumen extending within the second mandrel 220. The second guidewire 240 may be held and/or maintained in a substantially constant position within the second lumen of the second mandrel 220 until the physician is ready to use it.

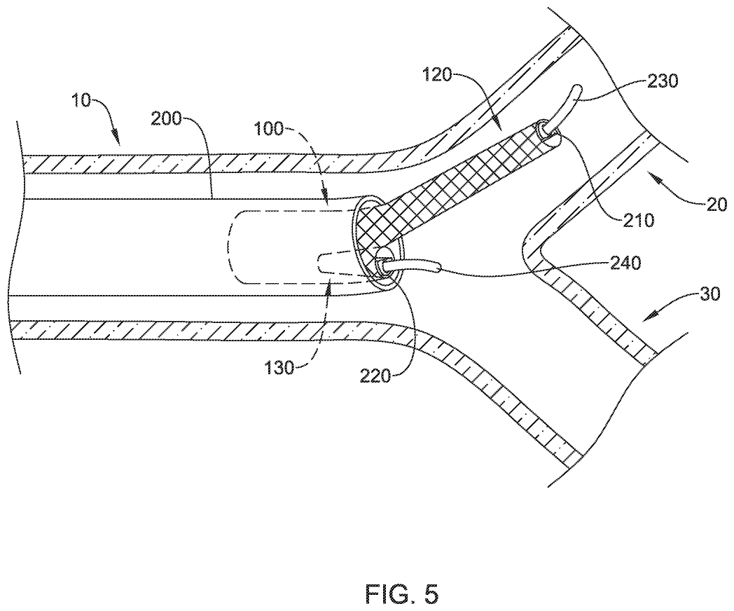

After tracking the first mandrel 210 and the elongate shaft 200 into the first branch lumen 20, the first mandrel 210 may be held in a constant position as the elongate shaft 200 (and the second mandrel 220 disposed therein) is withdrawn proximally to expose the first leg 120 of the stent 100 within the first branch lumen 20, as seen in FIG. 5. In some embodiments, the first mandrel 210 may include a distally facing shoulder configured to engage the proximal end 112 of the main body portion 110 of the stent 100. The distally facing shoulder of the first mandrel 210 may be configured to prevent proximal translation of the stent 100 relative to the first mandrel 210 and/or to urge the stent 100 out of the lumen of the elongate shaft 200 as the elongate shaft 200 is translated and/or withdrawn proximally relative to the first mandrel 210. In some embodiments, the first leg 120 of the stent 100 may begin to radially expand toward the deployed configuration after the first leg 120 has been exposed from the lumen of the elongate shaft 200.

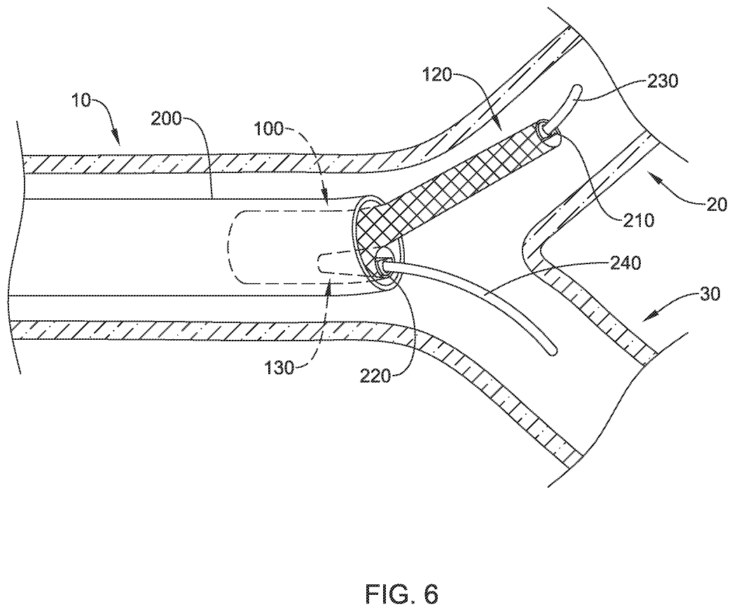

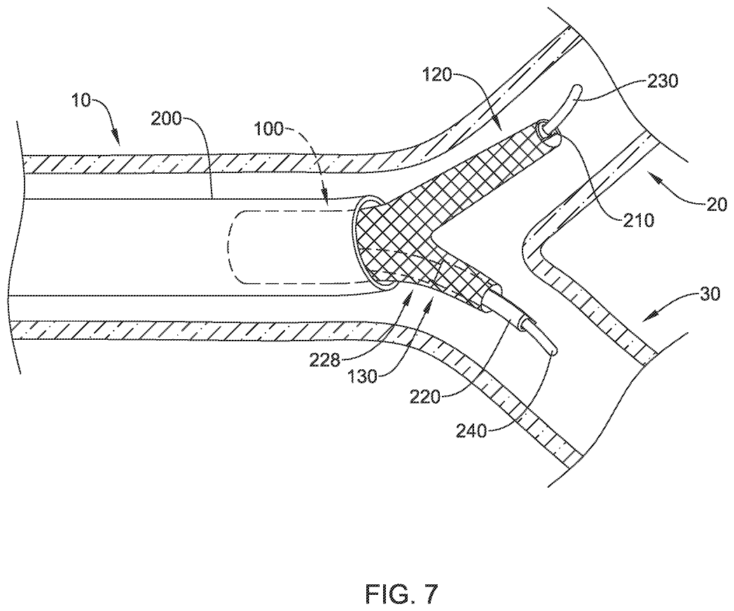

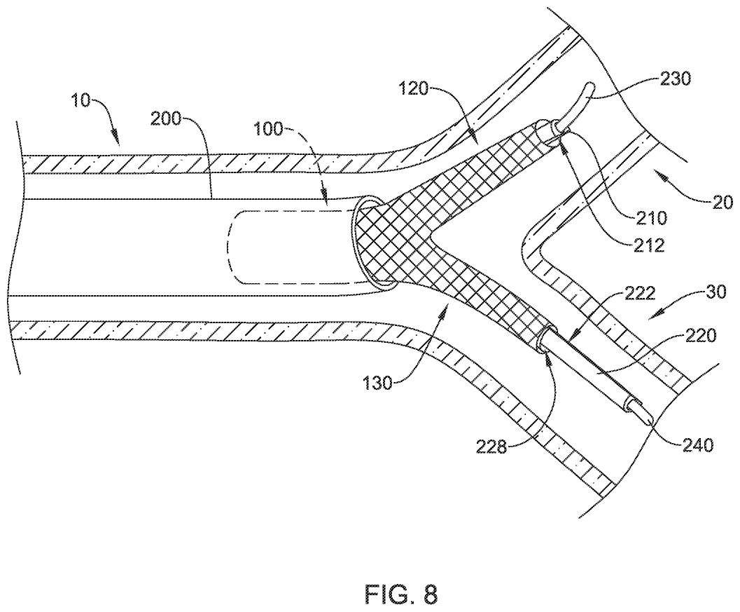

Next, the second guidewire 240 may be extended out of the second lumen of the second mandrel 220 and advanced into the second branch lumen 30, as shown in FIG. 6. Thereafter, the second mandrel 220 may be advanced and/or tracked over the second guidewire 240 into the second branch lumen 30 to evert the second leg 130 of the stent 100 within the second branch lumen 30, as shown in FIGS. 7-8. The second mandrel 220 may be configured to shift the stent 100 from the delivery configuration toward the deployed configuration by everting the second leg 130 of the stent 100. In some embodiments, the second mandrel 220 may include a distally facing shoulder 228 along the distal portion of the second mandrel 220. The distally facing shoulder 228 of the second mandrel 220 may be configured to engage the second end 134 of the second leg 130 of the stent 100 in the delivery configuration such that distal advancement of the second mandrel 220 relative to the main body portion 110 of the stent 100 everts the second leg 130 to shift the stent 100 toward the deployed configuration. As such, as the second mandrel 220 is advanced distally relative to the main body portion 110 of the stent 100 and/or the elongate shaft 200, the distally facing shoulder 228 pushes the inverted second leg 130 distally from inside the main body portion 110 of the stent 100 out into the second branch lumen 30, thereby everting the second leg 130.

As may be seen in FIG. 8, the distal portion of the second mandrel 220 having the D-shaped cross-section may include a first portion and a second portion disposed proximal of the first portion. The distally facing shoulder 228 may be disposed at a distal end of the second portion and/or a proximal end of the first portion. The second portion may have a greater cross-sectional area than first portion to facilitate the distally facing shoulder 228 engaging the proximal end 112 of the main body portion 110 of the stent 100.

Next, the elongate shaft 200 may be translated and/or withdrawn proximally relative to the stent 100 to completely release the stent 100 and permit the stent 100 to shift to a fully expanded configuration. In some embodiments, at least the first mandrel 210 may be held in a constant position as the elongate shaft 200 is translated and/or withdrawn proximally relative to the stent 100 to prevent the stent 100 from translating proximally along with the elongate shaft 200. In some embodiments, the second mandrel 220 may include a second distally facing surface configured to engage the proximal end 112 of the main body portion 110 of the stent 100 similar to the distally facing surface of the first mandrel 210.

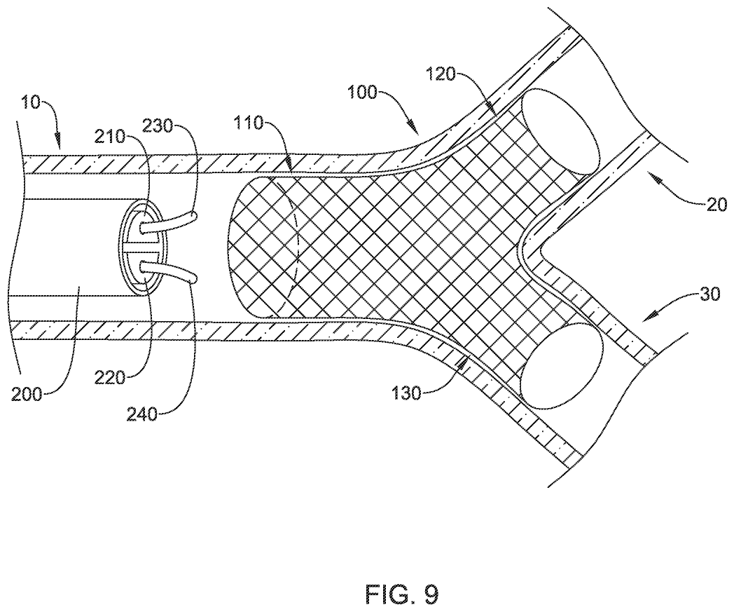

After the stent 100 has expanded radially to engage the wall of the body lumen 10, the first branch lumen 20, and the second branch lumen 30, the first mandrel 210, the second mandrel 220, the first guidewire 230, and the second guidewire 240 may be withdrawn into the lumen of the elongate shaft 200, as seen in FIG. 9. Thereafter, the elongate shaft 200 (and the components disposed therein) may be withdrawn and/or removed from the body lumen 10, leaving the stent 100 in place at the bifurcation.

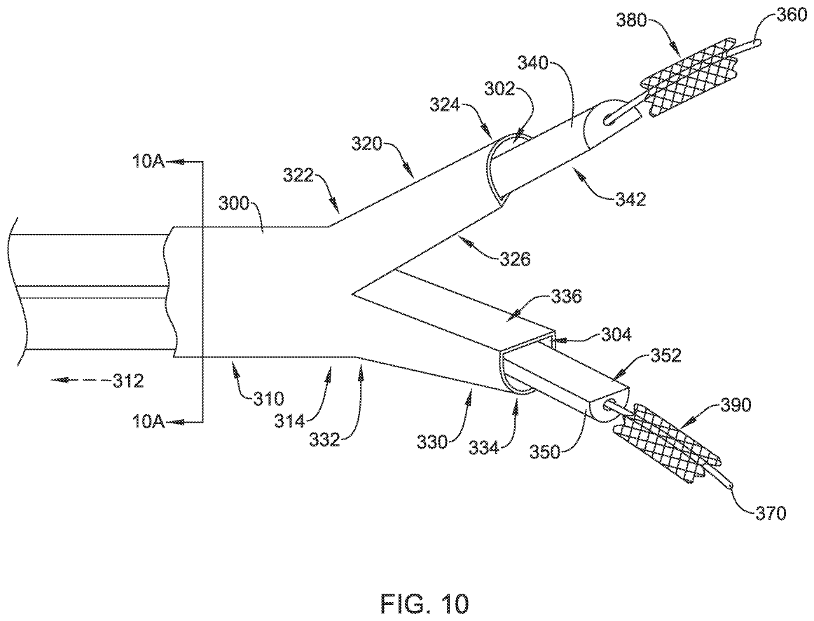

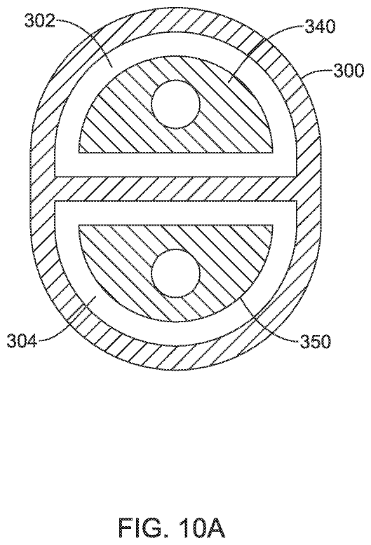

FIG. 10 illustrates selected aspects of a stent system comprising a delivery sheath 300 including a main body portion 310 having a proximal end 312 and a distal end 314, a first leg 320 having a first end 322 fixedly attached to the distal end 314 of the main body portion 310 and a second end 324 opposite the first end 322, and a second leg 330 having a first end 332 fixedly attached to the distal end 314 of the main body portion 310 and a second end 334 opposite the first end 332. In some embodiments, the delivery sheath 300 may be considered and/or may be referred to as a split sheath or a bifurcated sheath.

In some embodiments, the second end 324 of the first leg 320 and the second end 334 of the second leg 330 may be biased apart and/or away from each other laterally with respect to a longitudinal axis of the main body portion 310. In some embodiments, the second end 324 of the first leg 320 and the second end 334 of the second leg 330 may be self-biased apart and/or away from each other laterally with respect to a longitudinal axis of the main body portion 310. Other configurations are also contemplated.

In some embodiments, the delivery sheath 300 may include a first mandrel 340 slidably disposed within the first leg 320 of the delivery sheath 300. In some embodiments, the first mandrel 340 may be slidably disposed within the first leg 320 of the delivery sheath 300 and the main body portion 310 of the delivery sheath 300. In some embodiments, the delivery sheath 300 may include a second mandrel 350 slidably disposed within the second leg 330 of the delivery sheath 300. In some embodiments, the second mandrel 350 may be slidably disposed within the second leg 330 of the delivery sheath 300 and the main body portion 310 of the delivery sheath 300.

In some embodiments, the delivery sheath 300 may include a first guidewire 360 slidably disposed within the first mandrel 340 and/or the first leg 320 of the delivery sheath 300. In some embodiments, the first guidewire 360 may be slidably disposed within the first mandrel 340 and the first leg 320 and/or the main body portion 310 of the delivery sheath 300. In some embodiments, the delivery sheath 300 may include a second guidewire 370 slidably disposed within the second mandrel 350 and/or the second leg 330 of the delivery sheath 300. In some embodiments, the second guidewire 370 may be slidably disposed within the second mandrel 350 and the second leg 330 and/or the main body portion 310 of the delivery sheath 300.

In some embodiments, the delivery sheath 300 may include a first lumen 302 extending from the proximal end 312 of the main body portion 310 to the second end 324 of the first leg 320 and a second lumen 304 extending from the proximal end 312 of the main body portion 310 to the second end 334 of the second leg 330. In some embodiments, the first lumen 302 has a D-shaped cross-section having a first flat side. In some embodiments, the second lumen 304 has a D-shaped cross-section having a second flat side.

In some embodiments, the first mandrel 340 has a D-shaped cross-section having a first flat side 342. In some embodiments, the second mandrel 350 has a D-shaped cross-section having a second flat side 352. In some embodiments, the first flat side 342 of the first mandrel 340 may face toward the second flat side 352 of the second mandrel 350. In some embodiments, the first flat side 342 of the first mandrel 340 may face and/or align with the first flat side of the first lumen 302. In some embodiments, the first flat side 342 of the first mandrel 340 and the first flat side of the first lumen 302 prevent relative rotation of the first mandrel 340 within the first lumen 302 while permitting axially translation and/or sliding of the first mandrel 340 within the first lumen 302. In some embodiments, the second flat side 352 of the second mandrel 350 may face and/or align with the second flat side of the second lumen 304. In some embodiments, the second flat side 352 of the second mandrel 350 and the second flat side of the second lumen 304 prevent relative rotation of the second mandrel 350 within the second lumen 304 while permitting axially translation and/or sliding of the second mandrel 350 within the second lumen 304. In some embodiments, at least a portion of the first lumen 302 and at least a portion of the second lumen 304 share a common wall defining both the first flat side and the second flat side, as seen in FIG. 10A. In some embodiments, at least a portion of the first lumen 302 and at least a portion of the second lumen 304 share a common wall defining both the first flat side and the second flat side within the main body portion 310 of the delivery sheath 300. For clarity, the first guidewire 360 and the second guidewire 370 are not shown in FIG. 10A.

Returning to FIG. 10, the first leg 320 of the delivery sheath 300 may include a flat side 326 and the second leg 330 of the delivery sheath 300 may include a flat side 336 facing toward the flat side 326 of the first leg 320. The flat side 326 of the first leg 320 and the flat side 336 of the second leg 330 may be complimentary and/or may be configured to matingly engage each other when the first leg 320 and the second leg 330 are constrained within a lumen to reduce the overall cross-section of the lumen required to accommodate the delivery sheath 300.

In some embodiments, the stent system may include a first stent 380 disposable within the first leg 320 and/or the first lumen 302 distal of the first mandrel 340. The first mandrel 340 may include a first distal face configured to engage the first stent 380. The first mandrel 340 may be configured to push the first stent 380 out of the first leg 320 and/or the first lumen 302 via axial translation of the first mandrel 340 relative to the first leg 320. In one example, the delivery sheath 300 may be held in a substantially constant position while the first mandrel 340 is advanced distally within the first lumen 302 and/or the first leg 320. In another example, the first mandrel 340 may be held in a substantially constant position while the delivery sheath 300 is retracted proximally over the first mandrel 340. Other examples, including combinations thereof, are also contemplated.

In some embodiments, the stent system may include a second stent 390 disposable within the second leg 330 distal of the second mandrel 350. The second mandrel 350 may include a second distal face configured to engage the second stent 390. The second mandrel 350 may be configured to push the second stent 390 out of the second leg 330 and/or the second lumen 304 via axial translation of the second mandrel 350 relative to the second leg 330. In one example, the delivery sheath 300 may be held in a substantially constant position while the second mandrel 350 is advanced distally within the second lumen 304 and/or the second leg 330. In another example, the second mandrel 350 may be held in a substantially constant position while the delivery sheath 300 is retracted proximally over the second mandrel 350. Other examples, including combinations thereof, are also contemplated.

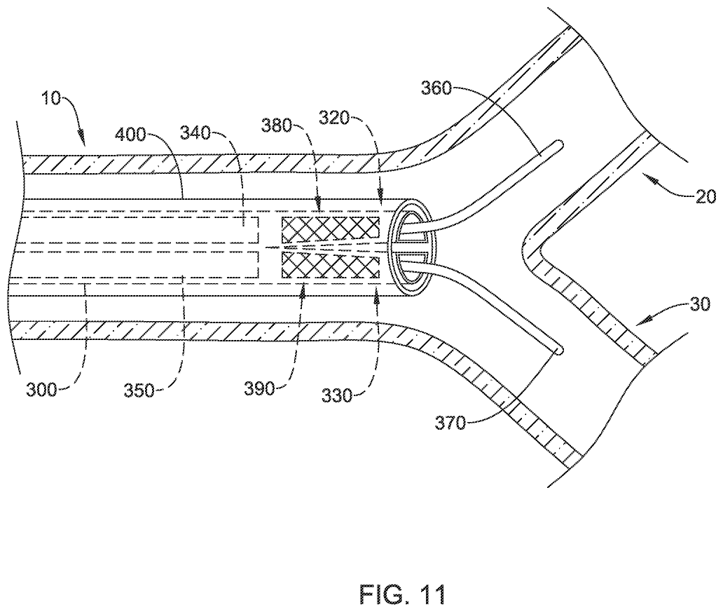

In FIG. 11, the stent system is illustrated within the body lumen 10. As seen in FIG. 11, the stent system may include an elongate shaft 400 having a lumen extending therein. The delivery sheath 300 may be disposable and/or is disposed within the lumen of the elongate shaft 400 and is axially slidable with respect to the elongate shaft 400 and/or within the lumen of the elongate shaft 400. In use, the stent system may be advanced into and/or within the body lumen 10 toward the bifurcation or Y-junction of the body lumen 10. As shown, the first guidewire 360 may be advanced and/or placed into the first branch lumen 20 and/or the second guidewire 370 may be advanced and/or placed into the second branch lumen 30. In some embodiments, the first guidewire 360 and the second guidewire 370 may be advanced and/or placed in sequence. In some embodiments, the first guidewire 360 and the second guidewire 370 may be advanced and/or placed simultaneously. The stent system permits the placement of both the first guidewire 360 and the second guidewire 370 using a single access point and/or device, thereby reducing opportunities for the introduction of air bubbles into the patient, the body lumen 10, and/or the stent system (and/or components thereof). In some embodiments, the first guidewire 360 and the second guidewire 370, following their initial placement into the first branch lumen 20 and the second branch lumen 30, respectively, may be disposed within the patient and usable for guiding and/or tracking the first and second mandrels, the first and second legs, etc. at the same time.

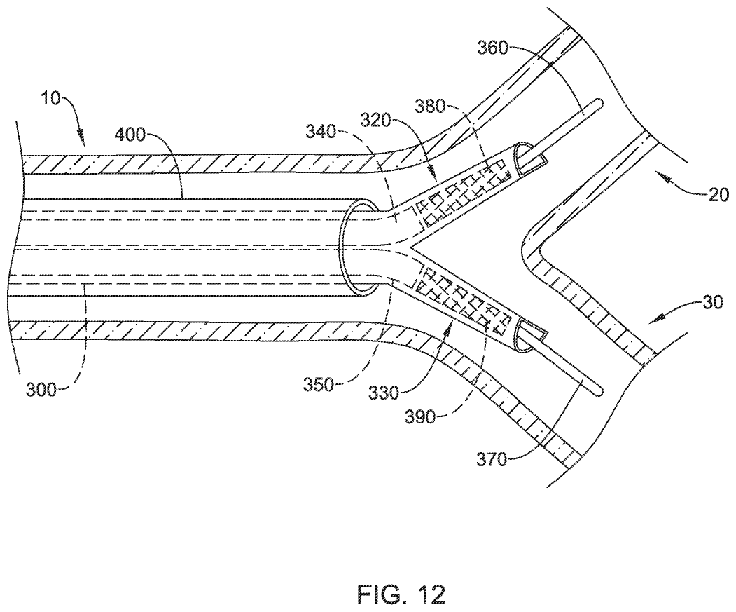

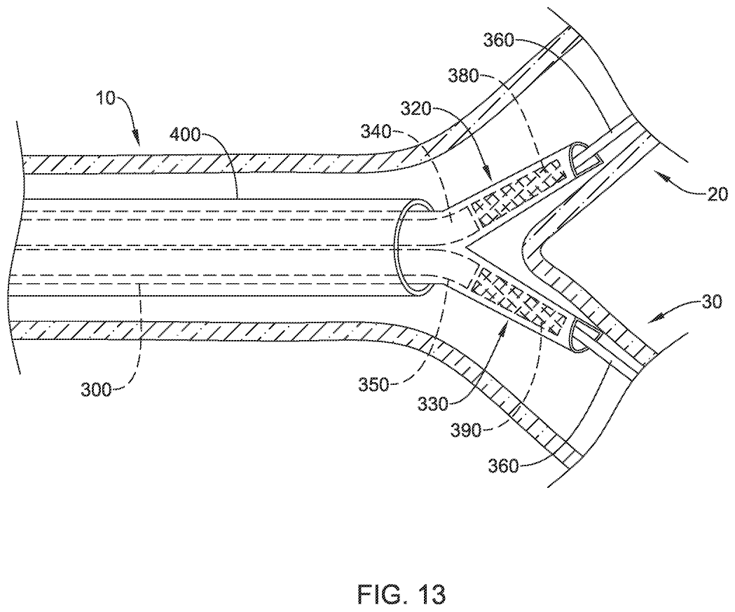

After placement of the first guidewire 360 into the first branch lumen 20 and the second guidewire 370 into the second branch lumen 30, the delivery sheath 300 may be advanced out of the lumen of the elongate shaft 400 distally within the body lumen 10 such that the first leg 320 advances over the first guidewire 360 into the first branch lumen 20 and the second leg 330 advanced over the second guidewire 370 into the second branch lumen 30, as seen in FIGS. 12-13. The first stent 380 may be disposed within the first leg 320 and the second stent 390 may be disposed within the second leg 330. As such, the first stent 380 is advanced into first branch lumen 20 along with the first leg 320 and the second stent 390 is advanced into the second branch lumen 30 along with the second leg 330. As the delivery sheath 300 is advanced distally out of the lumen of the elongate shaft 400, the first leg 320 and the second leg 330 may be biased apart and/or away from each other laterally, which will make it easier for the first leg 320 to track over the first guidewire 360 into the first branch lumen 20 and the second leg 330 to track over the second guidewire 370 into the second branch lumen 30. Relative movement and/or axial translation between the elongate shaft 400 and the delivery sheath 300 may be used to control the spread of the first leg 320 and the second leg 330. For example, as more of the delivery sheath 300 (e.g., the first leg 320 and the second leg 330) is exposed from and/or advanced distally relative to the elongate shaft 400, the first leg 320 and the second leg 330 may spread farther apart laterally.

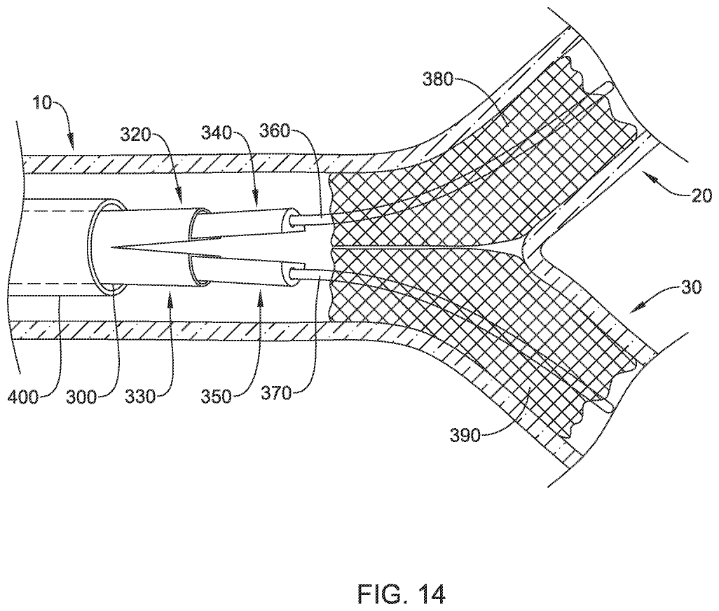

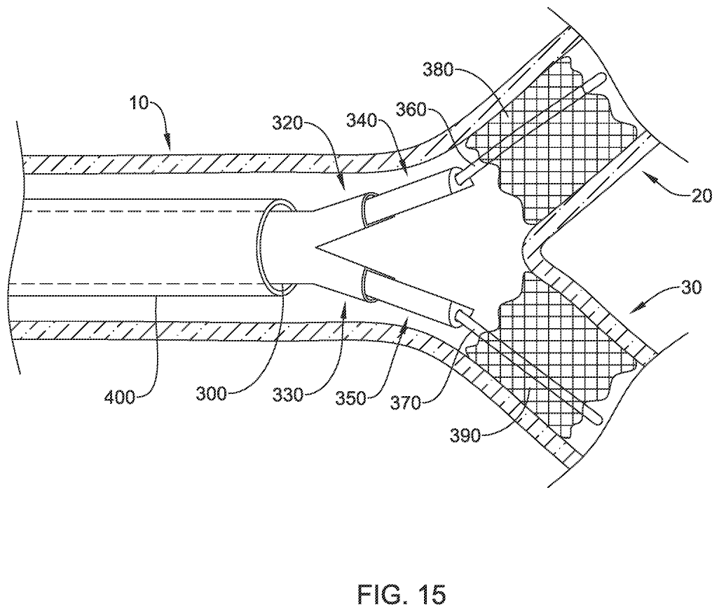

Thereafter, the first mandrel 340 and the second mandrel 350 may be held in a substantially constant position while the delivery sheath 300 and/or the elongate shaft 400 is withdrawn proximally relative to the first mandrel 340 and the second mandrel 350 to push the first stent 380 distally out of the first leg 320 and/or the first lumen 302 into the first branch lumen 20 and/or the body lumen 10 and to push the second stent 390 distally out of the second leg 330 and/or the second lumen 304 into the second branch lumen 30 and/or the body lumen 10 to deploy the first stent 380 and the second stent 390. In some embodiments, the first stent 380 may be positioned partially within the first branch lumen 20 and partially within the body lumen 10 and the second stent 390 may be positioned partially within the second branch lumen 30 and partially within the body lumen 10 alongside the first stent 380, as shown in FIG. 14. In some embodiments, the first stent 380 may be positioned completely within the first branch lumen 20 and the second stent 390 may be positioned completely within the second branch lumen 30, as seen in FIG. 15. Other configurations and/or positioning are also contemplated.

The stent system and the delivery sheath 300 may be useful for deploying the first stent 380 and the second stent 390 simultaneously at, across, and/or adjacent to a bifurcation in the body lumen 10. By introducing all of the necessary elements in a single device or system, fewer exchanges are needed and/or opportunities for the introduction of air bubbles into the system, the patient, the body lumen 10, etc. are reduced, thereby improving physician visibility and patient safety.

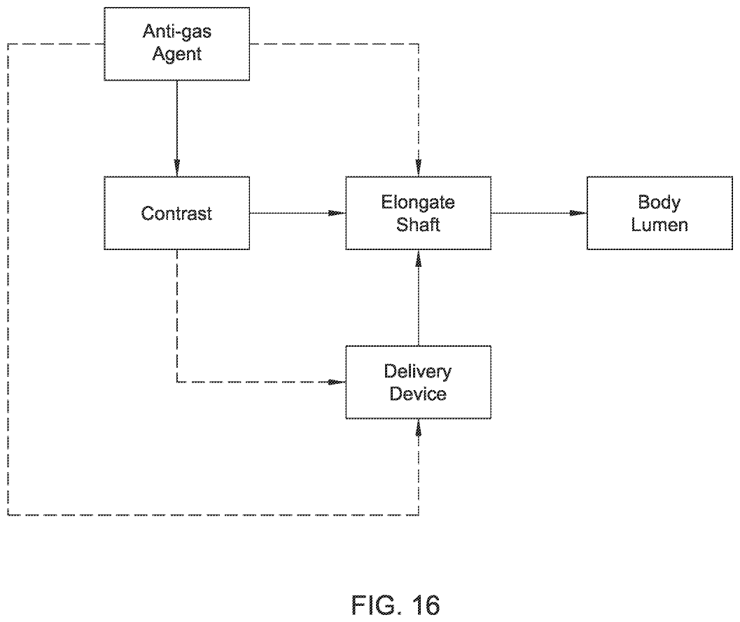

FIG. 16 is a block diagram illustrating selected aspects of a stent system configured to reduce air or gas bubbles introduced into a body lumen. In some embodiments, a stent system may include an elongate shaft configured to access a body lumen of a patient. The elongate shaft may include a lumen extending therein. In some embodiments, the elongate shaft may be an endoscope or an endoscopic device having at least one lumen disposed therein. Other configurations are also contemplated. In some embodiments, the stent system may include a delivery device slidably disposed within the lumen of the elongate shaft. The delivery device may be configured to deliver a stent to the body lumen. In some embodiments, the delivery device may be configured to deliver a bifurcated stent to the body lumen and/or to a bifurcation of the body lumen. The stent system may include a source of contrast fluid in fluid communication with the lumen of the elongate shaft for delivery of the contrast fluid to the body lumen. The contrast fluid may be used to aid in delivery and/or placement of the stent as is known in the art. In at least some embodiments, the contrast fluid may include an anti-gas agent, such as but not limited to simethicone. Other anti-gas agents are also contemplated.

In some embodiments, the anti-gas agent may optionally be delivered directly into and/or through the elongate shaft and/or the lumen of the elongate shaft to the body lumen. In some embodiments, the anti-gas agent may optionally be delivered directly into and/or through the delivery device to the body lumen. In some embodiments, the source of contrast fluid may optionally be in fluid communication with the delivery device instead of the lumen of the elongate shaft. In such embodiments, the contrast fluid may be delivered to the body lumen by the delivery device instead of the elongate shaft.

A method of treating a body lumen may include accessing the body lumen of a patient with an elongate shaft having a lumen extending therein. As discussed herein, in some embodiments, the elongate shaft may be an endoscope or an endoscopic device. Other configurations and/or devices are also contemplated. The method may include inserting a delivery device, such as but not limited to any of the devices described herein, within the lumen of the elongate shaft. The delivery device may be configured to deliver a stent to the body lumen. In some embodiments, the delivery device may be configured to deliver a bifurcated stent to the body lumen or to a bifurcation of the body lumen. In at least some embodiments, the method may further include delivering a contrast fluid including an anti-gas agent into the elongate shaft, the delivery device, and/or the body lumen while implanting the stent within the body lumen. In some embodiments, the method may include delivering a contrast fluid including an anti-gas agent while implanting a bifurcated stent within the body lumen and/or at or within a bifurcation of the body lumen. In some embodiments, the method may include injecting the anti-gas agent into the contrast fluid before delivering the contrast fluid to the body lumen. In some embodiments, the method may include injecting the anti-gas agent into the contrast fluid as the contrast fluid is being delivered to the body lumen. In some embodiments the anti-gas agent may be comingled and/or mixed within the contrast fluid. In some embodiments, the anti-gas agent may be dissolved within the contrast fluid.

The materials that can be used for the various components of the stent system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the elongate shaft, the mandrel(s), the guidewire(s), the polymeric cover, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol, can be used to achieve desired properties.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system and/or components thereof in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or components thereof to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent system, comprising:
a stent configured to shift between a delivery configuration and a deployed configuration, the stent comprising:
a main body portion having a proximal end and a distal end;
a first leg having a first end fixedly attached to the distal end of the main body portion and a second end opposite the first end, the first leg extending distally from the distal end of the main body portion in the deployed configuration; and
a second leg having a first end fixedly attached to the distal end of the main body portion and a second end opposite the first end, the second leg extending distally from the distal end of the main body portion in the deployed configuration;
wherein the second leg extends proximally from the distal end of the main body portion in the delivery configuration.

2. The stent system of claim 1, wherein the second leg is inverted into the main body portion such that the second leg extends proximally from the distal end of the main body portion within the main body portion in the delivery configuration.

3. The stent system of claim 1, further comprising an elongate shaft having a lumen extending therein;
wherein the stent is disposable within the lumen in the delivery configuration, the stent being configured to shift from the delivery configuration to the deployed configuration when the stent is disposed outside of the lumen.

4. The stent system of claim 3, further comprising:
a first mandrel slidably disposed within the lumen; and
a second mandrel slidably disposed within the lumen alongside the first mandrel;
wherein the first mandrel is at least partially disposed within the first leg and the second mandrel is at least partially disposed within the second leg.

5. The stent system of claim 4, wherein a first guidewire is slidably disposed within a first lumen extending within the first mandrel.

6. The stent system of claim 4, wherein a second guidewire is slidably disposed within a second lumen extending within the second mandrel.

7. The stent system of claim 4, wherein the second mandrel is configured to shift the stent from the delivery configuration toward the deployed configuration.

8. The stent system of claim 7, wherein the second mandrel includes a distally facing shoulder configured to engage the second end of the second leg in the delivery configuration such that distal advancement of the second mandrel everts the second leg to shift the stent toward the deployed configuration.

9. The stent system of claim 4, wherein a distal portion of the first mandrel has a D-shaped cross-section having a first flat side, a distal portion of the second mandrel has a D-shaped cross-section having a second flat side, and the first flat side faces the second flat side within the lumen of the elongate shaft.

10. The stent system of claim 1, wherein the first leg is tapered radially inward from the first end toward the second end.

11. The stent system of claim 1, wherein the second leg is tapered radially inward from the first end toward the second end.

12. A stent system, comprising:
a delivery sheath including a main body portion having a proximal end and a distal end, a first leg having a first end fixedly attached to the distal end of the main body portion and a second end opposite the first end, and a second leg having a first end fixedly attached to the distal end of the main body portion and a second end opposite the first end;
a first mandrel slidably disposed within the first leg of the delivery sheath;
a second mandrel slidably disposed within the second leg of the delivery sheath;
a first guidewire slidably disposed within the first mandrel; and
a second guidewire slidably disposed within the second mandrel.

13. The stent system of claim 12, wherein the delivery sheath includes a first lumen extending from the proximal end of the main body portion to the second end of the first leg and a second lumen extending from the proximal end of the main body portion to the second end of the second leg.

14. The stent system of claim 13, wherein the first lumen has a D-shaped cross-section having a first flat side and the second lumen has a D-shaped cross-section having a second flat side.

15. The stent system of claim 14, wherein the first lumen and the second lumen share a common wall defining both the first flat side and the second flat side.

16. The stent system of claim 13, wherein the first leg has a flat side, and the second leg has a flat side facing toward the flat side of the first leg.

17. The stent system of claim 16, further comprising an elongate shaft having a lumen extending therein;
wherein the delivery sheath is disposable within the lumen of the elongate shaft and axially slidable with respect to the elongate shaft;
wherein the flat side of the first leg matingly engages the flat side of the second leg when the first leg and the second leg are disposed within the lumen of the elongate shaft.

18. The stent system of claim 12, further comprising:
a first stent disposable within the first leg distal of the first mandrel; and
a second stent disposable within the second leg distal of the second mandrel;
wherein the first mandrel is configured to push the first stent out of the first leg via axial translation of the first mandrel relative to the first leg;
wherein the second mandrel is configured to push the second stent out of the second leg via axial translation of the second mandrel relative to the second leg.

19. The stent system of claim 12, wherein the second end of the first leg and the second end of the second leg are biased away from each other laterally with respect to a longitudinal axis of the main body portion.

20. A method of treating a body lumen, comprising:
accessing a body lumen of a patient with an elongate shaft having a lumen extending therein;
inserting a delivery device within the lumen of the elongate shaft, the delivery device being configured to deliver a stent to the body lumen; and
delivering a contrast fluid including an anti-gas agent while implanting the stent within the body lumen.
